(19) United States
(12) Patent Application Publication
(10) Pub. No.: US 2020/0146893 A1
(43) Pub. Date: May 14, 2020

(54) KIT FOR PROTECTING A SKIN SURFACE OF A PATIENT, INCORPORATING AT LEAST ONE IDENTIFICATION SYSTEM

(71) Applicant: DOUCHOSSEC, Aubagne (FR)

(72) Inventors: Eric GILLI, Cassis (FR); Jérôme STUBLER, PARIS (FR)

(21) Appl. No.: 16/615,860

(22) PCT Filed: May 25, 2018
(86) PCT No.: PCT/EP2018/000274

§ 371 Date: Nov. 22, 2019

(30) Foreign Application Priority Data
May 25, 2017 (FR) 1770538

(51) Int. Cl. A61F 13/00; A61M 25/02; A61F 15/00; G16H 10/65; G16H 20/10

(52) U.S. Cl. CPC A61F 13/00051; A61M 25/02; A61F 15/005; G16H 10/65; A61M 2205/6009; A61M 2025/0246; A61M 2025/0266; A61M 2205/6054; A61M 2205/6072; G16H 20/10

(57) ABSTRACT

The present invention relates to a kit for protecting a skin surface of a patient comprising at least one cover sheet, which is thin and flexible, and is intended to be applied on this skin surface and at least one packaging containing an adhesive substance and having an outlet port for applying a bead of this adhesive substance which locally connects the skin of the patient and the cover sheet; which at least one cover sheet is associated with at least one first identification system carrying information corresponding to at least one first unique identifier, which system can take the form of an electronic chip, a radio frequency tag (RFID support), a barcode or a QR code; and which kit further comprises a first reading device specific to this first identification system and which makes it possible to have access to the information of which it is a carrier.

The present patent application claims the priority of the French patent application FR 17/70538 filed on May 25, 2017 and which is incorporated in the present patent application by reference.

The present invention relates to a kit for protecting a skin surface of a patient.

It is known from the international application WO 2006/100382, a device for protecting a skin surface.

It is also known from the international application WO 2015/140454, a device for ensuring the maintenance and protection of a catheter on an area of the body of a patient.

Today, one of the challenges of the present and future medical world is to deal with medicines and medical devices counterfeit. Thus, it becomes necessary to be able to ensure the veracity of these. Another challenge is to be able to ensure the traceability of medicines and medical devices.

To solve these problems, a first object of the invention is a kit for protecting a skin surface of a patient comprising:
At least one cover sheet, which is thin and flexible, and is intended to be applied on this skin surface; and
at least one packaging containing an adhesive substance and having an outlet port allowing the setting up of a bead of this adhesive substance which locally connects the skin of the patient and the cover sheet;

Which kit is characterized in that:
said at least one cover sheet is associated with at least one first identification system carrying an information corresponding to at least a first unique identifier, which system can take the form of an electronic chip, a radio frequency tag (RFID), a barcode or a QR code; and
it further comprises a first reading device specific from this first identification system and which allows access to the information it carries.

The patient skin surface to be protected may be a skin surface subjected to inflammation, burning or even rupture (e.g. scratching). As such, this skin surface can be already covered with a dressing.

The cover sheet is preferably made of a non-allergenic material, such as polyurethane or a polyurethane-based polymer. Now, this cover sheet may be transparent or opaque, preferably transparent so as to easily observe the skin surface condition without having to remove it.

In order to fulfill its role of patient skin surface protection, this cover sheet is naturally waterproof.

The cover sheet thickness should be sufficient, depending on its composition, to allow good sealing and good impact resistance. Typically, this thickness should be comprised between 2 micrometers and 2 millimeters, preferably between 5 and 500 microns and, particularly preferably, between 10 microns and 80 microns.

Advantageously, the cover sheet dimensions will be such that it can cover a wide range of skin surfaces. To do so, the cover sheet may be cut, in particular by means of a pair of scissors, provided that care is taken to maintain at least a first identification system in the cut portion intended to be applied.

For example, the cover sheet may have a surface comprised between 5 and 500 cm², preferably between 10 and 100 cm².

In connection with the first identification system, which is a critical element of the invention, it may take the form of an electronic chip, a radio frequency tag (RFID support), a barcode or a QR code.

According to a first preference, the first identification system will take the form of an electronic chip or a radio frequency tag and, particularly preferably, a radio frequency tag.

According to a second preference, the first identification system will take the form of a barcode or a QR code, preferably a QR code.

This first identification system may be adhered to one of the faces of the at least one cover sheet, preferably on the inner face of the at least one cover sheet (the one in contact with the skin surface of the patient) or be isolated between at least two layers of materials within or not of said at least one cover sheet.

This first identification system will have a weight such that it does not constitute a constraint for the holding of the cover sheet. Typically, its weight is less than 10 grams, preferably less than 5 grams, and particularly preferably less than one gram.

When the first identification system is isolated between two layers of materials, one of the two material layers may correspond to the cover sheet and the second layer to a carrier sheet. This carrier sheet is then distinct from the cover sheet.

Ideally, this carrier sheet is positioned on the outer face of the cover sheet—i.e. the one which is not contacting the patient's skin-. It is preferably made of a flexible material, which may be similar to that of the cover sheet (e.g. polyurethane or polyurethane-based polymer) or not. The flexible nature of this material allows the carrier sheet to adapt as closely as possible to the shape modifications of the cover sheet so as not to risk affecting the integrity of the latter, piercing or tearing it. Now, and although flexible, the carrier sheet may be flat or have a curvature, which curvature can better adapt to that of the skin surface on which is applied the kit according to the invention. In this case, this curvature may make it possible to limit the stresses on the cover sheet once the kit of the invention placed on the skin of the patient.

Still related to the material of the cover sheet, this one may be transparent or opaque. In the case where the first identification system must be read (QR code or barcode), the carrier sheet will be in a transparent material for viewing and therefore reading this first identification system.

In connection with the carrier sheet's dimensions, these are such as to cover, and thus to protect, the entire first identification system. Now, and in order not to affect the flexibility of the cover sheet, and therefore of the kit according to the invention, the surface covered by the carrier sheet will be greater than the surface of the first identification system. Typically, the value of the surface of the carrier sheet is comprised between 1.5 and 10 times the value of the surface of the first identification system, preferably it is comprised between 2 and 5 times the value of the surface of the first system Identification.

Now and preferably, the shape of the carrier sheet has no stops so as not to risk altering the integrity of the cover sheet with which it is associated. Such a risk is indeed important, especially when laying on the skin of the patient, the protective kit according to the invention or, following movement of the patient. The carrier sheet may thus have an oval or round shape, preferably a round shape. One can also choose a square or rectangular shape, since the corners will be rounded. According to a particular embodiment, said at least one cover sheet may be associated with a detachable protective sheet applied against its inner face, the one that is intended to be applied on the skin surface. In addition, this detachable protective sheet will ideally have a lower flexibility than said cover sheet so as to facilitate its separation from the cover sheet before application of the latter.

In connection with this radio frequency tag (RFID support), it comprises in known manner an induction loop providing power to a circuit comprising storage means forming a memory in which is recorded at least a first unique identifier. This radio frequency tag or RFID support is intended to transmit information to the device, including said at least one unique identifier. By way of example, such a radio frequency tag can operate in the frequency band between 13 and 14 MHz

In connection with the information carried by the identification system, it is at least a first unique identifier which may correspond, for example, to the batch number of the at least one cover sheet.

Now, additional information may be brought (especially recorded when it is related to electronic chips or radio frequency tags) by this first identification system, such as the date of manufacture of the cover sheet, the expiry date the cover sheet or the composition of this cover sheet.

In addition, this identification system can be coupled to or associated with one or more sensors, such as pressure, temperature, humidity, odor, brightness sensors or even biosensors (microorganism sensors, particularly bacteria).

Concerning the packaging containing an adhesive substance, it can take many forms, it can take the form of a compressible tube or of a rigid cartridge, pressurized or not.

In the case where the packaging containing an adhesive substance takes the form of a compressible tube, it then comprises an extrusion head or a neck arranged, in a manner known per se, to allow the distribution of the adhesive substance so that that it forms a beads of homogeneous width and, preferentially, flexible.

The adhesive substance can take, for example, the form of a liquid, a foam or a gel, preferably of a gel.

Once applied, this adhesive substance allows the adhesion of said at least one cover sheet to the skin surface and guarantee the sealing of the volume thus delimited (the skin surface surrounded by adhesive substance and covered with at least one cover sheet).

This adhesive substance is also non-allergenic and biocompatible.

Ideally, this adhesive substance may exhibit lower skin adhesion properties than its adhesion properties to the cover sheet; so as to allow the removal of the cover sheet without pain or adhesive residue on the skin.

Still ideally, the adhesion properties of the adhesive substance are such that they allow the cover sheet to be held on the patient's skin for a period of several days.

This adhesive substance advantageously comprises a homopolymer or a copolymer. Ideally, this adhesive substance comprises a copolymer, preferably based on silicone, such as for example glue based on polydimethylsiloxane, which copolymer may further comprise functional groups.

According to another particular embodiment, the packaging may comprise a second identification system carrying information corresponding to at least a second unique identifier and the kit according to the invention will then also comprise a second reading device specific to this second identification system that allows access to the information it carries.

As previously, this second identification system can take the form of an electronic chip, a radio frequency tag (RFID support), a barcode or a QR code. Preferably, this second identification system takes the same form as the first identification system and the first and the second reading device are then one and the same reading device.

Said at least one second unique identifier may correspond, for example, to the batch number of this packaging containing this adhesive substance.

Now, said information may further comprise the date of manufacture of this packaging containing this adhesive substance, the expiry date of this packaging containing this adhesive substance or the composition of this adhesive substance.

In addition, this second identification means may be coupled or associated with one or more sensors, such as pressure sensors, temperature, humidity, brightness odors or biosensors (microorganism sensors, including bacteria).

In connection with the reading device, the skilled person is able to determine the one to use depending on the chosen identification system.

In the case of an identification system in the form of an electronic chip or an RFID tag, the reading device will include at least one power supply for activating the identification system. In the case of a radio frequency tag or RFID support, this activation will thus be effected by means of a magnetic field captured by the induction loop of the RFID support. The reading device then comprises a receiver in the form of an antenna intended to receive the electromagnetic signals emitted by the radio frequency tag (s) or RFID media when the latter (s) is or are powered.

Preferably, the reading device or devices further comprise a user interface for displaying the information stored in the identification system or systems. Note that the reading device is capable of compiling or associating the sequence of identification devices, thus creating tracking chains.

According to yet another particular embodiment, and in the case where the identification system or systems take the form of an electronic chip or an RFID tag, the specific reading device or devices then comprise at least one microcontroller intended to the management of said identification system or systems, which microcontroller is at the same time in charge of reading the information of this or these identification systems and of the modification thereof. This microcontroller is thus able to modify the information present on the first identification system so as to specify the applying day, on the skin surface, of said at least one cover sheet and, optionally, the deadline to which it is appropriate to change this at least one cover sheet. This microcontroller is still able to modify the information present on the second identification system so as to specify the date of opening of the packaging containing an adhesive substance.

According to one embodiment wherein the packaging containing an adhesive substance takes the form of a cartridge, preferably pressurized, the reading device is further associated with:
An association means making it possible to access the contents of the packaging, which association means comprises a valve allowing at the same time to control the opening and closing of the packaging, and
a nozzle for dispensing the adhesive substance so that it forms a beads of uniform width and, preferably, flexible.

According to a preferred embodiment, the kit is also intended for maintaining and protecting a catheter, which kit then comprises a cover sheet whose central part, on its internal face which is applied to the skin, is associated with a pad having a shaped cavity for housing a non-invasive portion (head or fitting) of said catheter.

The pad may be glued or welded to the cover sheet and preferably has a planar shape.

The pad can be made of any material having the robustness and flexibility necessary to simultaneously allowing a good maintenance of the catheter and a good comfort for the patient. For example, it can be made of viscose, silicone, polypropylene, sodium polyacrylate, cellulose, . . . .

The peripheral fastening portion of the cover sheet—i.e. the portion extending around the pad, preferably overflowing widely around the pad-. A large fastening part allows to choose with greater freedom the position of the adhesive gel bead. It is thus possible, if necessary, to bypass an injured or altered part of the patient's skin, if such an injured or impaired part is in the vicinity of the catheter. In addition, it is possible to avoid thus applying the adhesive substance on the pad. Now, it is possible to put a thin layer of adhesive on the inner face of the pad to facilitate its application to the skin and improve the immobilization of the catheter.

1. A kit for protecting a skin surface of a patient comprising:
At least one cover sheet, which is thin and flexible, and is intended to be applied on this skin surface; and
at least one packaging containing an adhesive substance and having an outlet port allowing the setting up of a bead of this adhesive substance which locally connects the skin of the patient and the cover sheet;
wherein:
said at least one cover sheet is associated with at least one first identification system carrying an information corresponding to at least a first unique identifier, which system can take the form of an electronic chip, a radio frequency tag (RFID), a barcode or a QR code; and
the kit further comprises a first reading device specific from this first identification system and which allows access to the information.

2. The kit of claim 1, wherein the first identification system takes the form of an electronic chip or of a radio frequency tag (RFID support).

3. The kit of claim 1, wherein the first identification system takes the form of a barcode or of a QR code.

4. The kit according to claim 1, wherein the first identification system is isolated between two layers of materials, one of which corresponding to the cover sheet and the other to a carrier sheet.

5. The kit of claim 4, wherein the carrier sheet is flat or has a curvature.

6. The kit of claim 4, wherein the carrier sheet is made of a transparent material.

7. The kit of claim 4, wherein the dimensions of the carrier sheet are sufficient so as to cover and thus protect the entire first identification system and with a value of the surface of the carrier sheet comprised between 1.5 and 10 times the value of the surface of the first identification system.

8. The kit of claim 4, wherein the shape of the carrier sheet has no stops.

9. The kit according to claim 1, wherein the packaging containing an adhesive substance can take the form of a compressible tube or of a rigid cartridge, pressurized or not.

10. The kit according to claim 1, wherein the packaging comprise a second identification system carrying information corresponding to at least a second unique identifier and the kit further comprises a second reading device specific to this second identification system that allows access to the information.

11. The kit according to claim 1, wherein the reading device or devices further comprise a user interface for displaying the information stored in the identification system or systems.

12. The kit according to claim 1, wherein the identification system or systems take the form of an electronic chip or an RFID tag, the specific reading device or devices then comprise at least one microcontroller intended to the management of said identification system or systems, which microcontroller is at the same time in charge of reading the information of this or these identification systems and of the modification thereof.

13. The kit according to claim 12, wherein the microcontroller is able to modify the information present:
on the first identification system so as to specify the applying day, on the skin surface, of said at least one cover sheet; and/or
on the second identification system so as to specify the date of opening of the packaging containing an adhesive substance.

14. The kit according to claim 1, wherein the packaging containing an adhesive substance takes the form of a cartridge, the reading device is further associated with:
an association means making it possible to access the contents of the packaging, which association means comprises a valve allowing at the same time to control the opening and closing of the packaging, and
a nozzle for dispensing the adhesive substance so that it forms a beads of uniform width.

15. The kit according to claim 1, wherein the kit is also intended for maintaining and protecting a catheter, which kit then comprises a cover sheet whose central part, on its internal face which is applied to the skin, is associated with a pad having a shaped cavity for housing a non-invasive portion (head or fitting) of said catheter.

16. The kit of claim 1, wherein the first identification system takes the form of a radio frequency tag (RFID support.

17. The kit of claim 1, wherein the first identification system takes the form of a QR code.

18. The kit according to claim 12, wherein the microcontroller is able to modify the information present:
on the first identification system so as to specify the applying day, on the skin surface, of said at least one cover sheet and the deadline to which it is appropriate to change this at least one cover sheet; and/or
on the second identification system so as to specify the date of opening of the packaging containing an adhesive substance.

19. The kit according to claim 1, wherein the packaging containing an adhesive substance takes the form of a pressurized cartridge, the reading device is further associated with:
an association means making it possible to access the contents of the packaging, which association means comprises a valve allowing at the same time to control the opening and closing of the packaging, and
a nozzle for dispensing the adhesive substance so that it forms flexible beads of uniform width.

20. The kit according to claim 2, wherein the first identification system is isolated between two layers of materials, one of which corresponding to the cover sheet and the other to a carrier sheet.
